(19) United States
(12) Patent Application Publication
(10) Pub. No.: US 2020/0316264 A1
(43) Pub. Date: Oct. 8, 2020

(54) RADIOPAQUE MATERIAL AND COMPOSITIONS AND DEVICES INCLUDING THE SAME

(71) Applicant: Medtronic, Inc., Minneapolis, MN (US)

(72) Inventor: Chandrasekhar Ramasubramanian Rajagopalan, New Brighton, MN (US)

(21) Appl. No.: 16/373,244

(22) Filed: Apr. 2, 2019

(51) Int. Cl. A61L 29/18; A61L 29/12; C08L 75/04; C08K 3/30; C08K 3/22; C08K 3/08

(52) U.S. Cl. CPC A61L 29/18; A61L 29/126; C08L 75/04; C08K 3/30; C08K 2201/00; C08K 3/08; C08K 2003/3045; C08K 2003/0887; C08K 3/22

(57) ABSTRACT

A radiopaque filler material contains a mixture of a first radiopaque material and a second radiopaque material different from the first radiopaque material. The radiopaque filler material may be dispersed in a polymeric material. The polymeric material may be used to prepare a medical device or a part thereof. The polymeric material containing the radiopaque filler material exhibits a level of radiopacity that is substantially even across varying imaging energy levels.

### FIELD

The present disclosure relates to radiopaque materials. The present disclosure further relates to compositions and medical devices that include radiopaque material. In particular, the present disclosure relates to radiopaque materials that include a mixture of two or more radiopaque materials and to compositions and medical devices including the same.

### BACKGROUND

It may be desirable to monitor the position of medical devices such as, e.g., catheters, leads, implants, etc., while they are within a patient's body. For example, it may be useful to monitor the position of guide catheters which are used to place catheters, electrode leads, and the like in desired locations within the body of a patient. A guide catheter typically includes an elongated sheath that is inserted into a blood vessel or another portion of the body. A catheter or lead is introduced through an inner channel defined by the sheath.

To enable precise positioning of a medical device, the medical device may include radiopaque material that is visible under fluoroscopy and/or X-ray imaging. Using fluoroscopic or X-ray imaging techniques, the physician can visualize the medical device to determine its position. For example, the physician may be able to visualize a guide catheter and place the catheter or electrode lead in a desired position.

The energy output of imaging devices varies from one device to another. It would be desirable to provide a radiopaque material that is visible in a wide variety of imaging devices and over a wide range of energy outputs.

### SUMMARY

A medical device includes a polymeric material and radiopaque filler material dispersed within the polymeric material. The radiopaque filler material may include a mixture of a first radiopaque material and a second radiopaque material different from the first radiopaque material. The first and second radiopaque materials may be selected from compounds containing barium (Ba), bismuth (Bi), tantalum (Ta), and/or tungsten (W).

A polymeric composition includes a polymer and radiopaque filler material dispersed within the polymer. The radiopaque filler material may include a mixture of a first radiopaque material and a second radiopaque material different from the first radiopaque material. The first and second radiopaque materials may be selected from compounds containing barium (Ba), bismuth (Bi), tantalum (Ta), and/or tungsten (W).

A polymeric composition includes a polymer and a means for imparting radiopacity to the polymeric composition. The polymeric composition may exhibit a first level of radiopacity at a first imaging energy output level and a second level of radiopacity at a second imaging energy output level, the second imaging energy output level being within 20 keV of the first imaging energy output level. The second level of radiopacity may be within 90% to 110% of the first level of radiopacity. The polymeric composition may exhibit the first and second levels of radiopacity throughout the polymeric composition.

The term “filler” is used in this disclosure to describe a material that is mixed into (e.g., dispersed throughout) a base material. For example, a filler may be mixed into a polymeric base material used to make an item, such as a medical device.

The term “tensile strength” is used in this disclosure to refer to the capacity of a material to withstand a pulling (tensile) force before the material breaks, tears, rips, etc.

The term “substantially” as used here has the same meaning as “nearly completely,” and can be understood to modify the term that follows by at least about 90%, at least about 95%, or at least about 98%. “The term “not substantially” as used herein can be understood to have the inverse meaning of “substantially,” i.e., modifying the term that follows by not more than 10%, not more than 5%, or not more than 2%, and in some instances may have the same meaning as “not significantly.”

The term “about” is used here in conjunction with numeric values to include normal variations in measurements as expected by persons skilled in the art, and is understood have the same meaning as “approximately” and to cover a typical margin of error, such as ±5% of the stated value.

Terms such as “a,” “an,” and “the” are not intended to refer to only a singular entity, but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list.

### BRIEF DESCRIPTION OF DRAWINGS

FIG. 1 is a graphical representation of variation of mass attenuation of four commonly used radiopaque elements.

FIG. 2 is a schematic view of a medical device according to an embodiment.

### DETAILED DESCRIPTION

The present disclosure relates to radiopaque filler materials that include a mixture of two or more radiopaque materials. The present disclosure further relates to polymeric compositions that include the radiopaque filler material. The present disclosure also relates to medical devices made of a polymeric material and the radiopaque filler material. The radiopaque materials in the mixture are selected to provide visibility across a spectrum of X-ray energy imaging levels.

Radiopaque materials are those that inhibit the passage of electromagnetic radiation, particularly X-rays. Using analog X-ray film, such materials have a whiter appearance compared with a relatively dark appearance of more radiolucent materials. Digital X-ray images usually appear inverted such that radiopaque components appear dark gray or black. To determine whether a particular medical device component, e.g., catheter, lead, or balloon, may be sufficiently radiopaque for the intended implant location in a human patient, a designer may utilize ASTM F640-12 “Standard Test Methods for Determining Radiopacity for Medical Use.”

The energy output of imaging devices varies and is typically in the range of 30-70 keV. Although imaging devices are usually operated at a fixed peak kilovoltage (kVp) setting, the energy output of a given device is a distribution rather than a single energy level. The kVp setting of the device varies and is often chosen automatically by the equipment software depending on the imaging location (that is, the body part, such as the spine, heart, etc.) and the imaging system (e.g., X-ray or fluoroscopy). Further, the energy output from one device to the next, even devices of the same type, make, and model, can vary. These factors result in significant variation in the energy output from one measurement to the next.

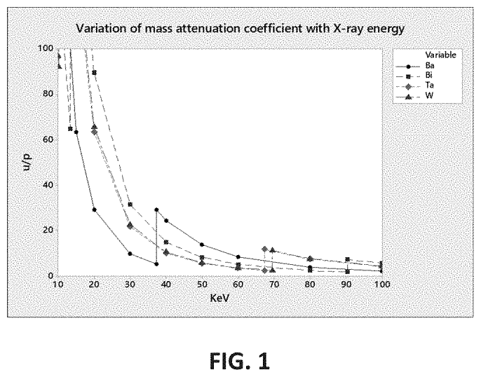

On the other hand, the ability of radiopaque materials to attenuate radiation varies sometimes significantly across the energy spectrum. The variation of mass attenuation coefficient of four elements commonly used in radiopaque materials, barium (Ba), bismuth (Bi), tantalum (Ta), and tungsten (W), as a function of X-ray energy is demonstrated in FIG. 1. As can be seen in the figure, the visibility of a radiopaque material may be excellent at one part of the energy spectrum (e.g., barium at 37 keV) and drop sharply at another part (e.g., barium at around 35 keV). Similar variations can be seen for tantalum and tungsten around 68-70 keV. As a result, users may sometimes experience issues with a medical device being visible at one energy level (for example in one imaging device or during one procedure), and not being sufficiently visible at another energy level (for example, in another imaging device or during another procedure).

Various embodiments of the present disclosure are directed to the use of multiple radiopaque filler materials in a manner that provides visibility across an energy spectrum. Particular embodiments can be useful for providing visibility at different energy levels that can occur with different imaging devices and techniques. For example, the visibility during imaging (e.g., the radiopacity) of the radiopaque filler materials of certain embodiments of the present disclosure is not significantly diminished within a given 20 keV range of the imaging device energy level (e.g., ranging from 30-50 keV, from 40-60 keV, or from 50-70 keV) or of the imaging device peak voltage level (e.g., ranging from 30-50 kVp, from 40-60 kVp, or from 50-70 kVp).

The level of radiopacity of a material may be expressed as a ratio of X-ray energy intensity that reaches the detector and the intensity of the incident X-ray energy. This is related to characteristics such as the mass attenuation coefficient, relative weight percent of ingredients, and the dimensions of the attenuating element. The level of radiopacity of two materials may be compared using an indirect measurement by comparing differences in the lightness or darkness of an image created with the imaging technique of interest (e.g., X-ray imaging). The images may be processed digitally, and the optical density or grayscale value of pixels may be averaged to yield a numeric value that may be used for comparison of the materials. The discussion below of relative radiopacities of materials refer to indirectly measured lightness or darkness unless otherwise indicated.

In some embodiments, the radiopaque filler material has a relatively constant level of radiopacity at different imaging energy output levels. The radiopaque filler material may have a first level of radiopacity at a first imaging energy output level and a second level of radiopacity at a second imaging energy output level. The first and second imaging energy output levels may be, for example, within about 10 keV, about 15 keV, about 20 keV, about 25 keV, or about 30 keV from each other. The first and second levels of radiopacity may be within 25%, 20%, 15%, 10%, or 5% from each other. The second level of radiopacity may be at least 75%, at least 80%, at least 85%, at least 90%, or at least 95% of the first level of radiopacity. The second level of radiopacity may be up to 125%, up to 120%, up to 115%, up to 110%, or up to 105% of the first level of radiopacity. In some embodiments, the first and second levels of radiopacity are substantially the same (e.g., are within 95-105% or one another) at any two imaging energy output levels that are within 20 keV from each other.

In some embodiments, the radiopaque filler material is mixed with one or more polymers to form a polymeric composition. The polymeric composition may have a first level of radiopacity at a first imaging energy level and a second level of radiopacity at a second imaging energy level. The first and second imaging energy levels may be, for example about 10 keV, about 15 keV, about 20 keV, about 25 keV, or about 30 keV from each other. The second level of radiopacity may be at least 75%, at least 80%, at least 85%, at least 90%, or at least 95% of the first level of radiopacity. The second level of radiopacity may be up to 125%, up to 120%, up to 115%, up to 110%, or up to 105% of the first level of radiopacity. In some embodiments, the first and second levels of radiopacity are substantially the same (e.g., are within 95-105% or one another) at any two imaging energy levels that are within 20 keV from each other. The polymeric composition may exhibit the first and second levels of radiopacity throughout the polymeric composition.

According to some embodiments, the radiopaque filler material includes a mixture of two or more radiopaque materials selected to provide similar (e.g., substantially even) visibility across the energy spectrum or across sections of the energy spectrum. In some embodiments, the mixture of two or more radiopaque materials is used as a filler in a material (e.g., polymeric material) used to prepare a medical device or a part of a medical device. In some embodiments, the mixture of two or more radiopaque materials is used as a filler in a material (e.g., polymeric material) used to prepare a radiopaque marker. It has been discovered that opacity provided by each of two or more radiopaque filler materials is not significantly diminished relative to the opacity of each radiopaque filler material with the same percentage of polymeric material. Stated another way, the opacity provided by each radiopaque filler material can substantially be maintained even after reducing the overall percentage of each individual filler material in order to account for the presence of multiple materials—while keeping the percentage of polymeric material the same. According to certain embodiments, this discovery can be particularly useful for constructing a polymeric device with opacity over a relatively wide range of energy levels.

Consistent with certain embodiments, the radiopaque filler materials can be selected to provide consistent opacity across energy levels corresponding to variations between the imaging devices and techniques. For example, the radiopaque filler materials can be selected to provide consistent opacity across energy levels where a single radiopaque material (such as barium around 35 keV to 37 keV or tantalum and tungsten around 68 keV to 70 keV) may cause significant variation in opacity. The radiopaque filler materials can be selected to provide consistent opacity at energy levels ranging from 30-50 keV (e.g., at 35 keV and 40 keV), from 40-60 keV (e.g., at 45 keV and 55 keV), or from 50-70 keV (e.g., at 65 keV and 75 keV). The radiopaque filler materials can be selected to provide consistent opacity from one device to another (e.g., from one X-ray imaging device to another X-ray imaging device) and from one type of imaging technique to another type of imaging technique (e.g., from X-ray imaging to fluoroscopic imaging).

The radiopaque filler material may be selected such that at a first imaging energy output level, one radiopaque material exhibits a higher level of radiopacity and another radiopaque material exhibits a lower level of radiopacity, and at a second imaging energy output level where the first radiopaque material exhibits a lower level of radiopacity, the other radiopaque material exhibits a higher level of radiopacity to compensate.

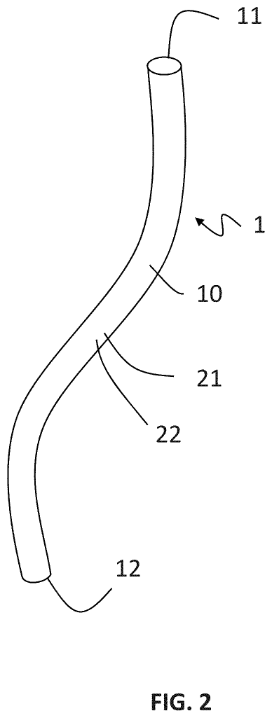

An exemplary medical device containing the radiopaque filler material is shown in FIG. 2. The medical device may be a catheter 1 having a body 10 that extends from a first end 11 to a second end 12. The body 10 may be made from a polymeric material 21 that includes a radiopaque filler material 22. The radiopaque filler material 22 may be mixed into (e.g., dispersed throughout) the polymeric material 21. The mixture of polymeric material 21 and radiopaque filler material 22 may extend throughout the body 10 from the first end 11 to the second end 12. In some embodiments, the mixture of polymeric material 21 and radiopaque filler material 22 only form a portion of the body 10, such as a tip, a section, a coating, or a band. The medical device (e.g., catheter 1) may include or be connected to other components or medical devices to facilitate its use or function.

The radiopaque filler material may include a mixture of two, three, four, or even more than four radiopaque materials. In some embodiments, the radiopaque filler material includes a mixture of two or three radiopaque materials. Any radiopaque material suitable for use as a filler may be selected. Preferably, the mixture of radiopaque materials includes materials selected to complement each other's opacity ranges such that one material exhibits stronger opacity in a range where another material may exhibit weaker opacity. For example, the materials may be selected such that at least one material exhibits relatively strong opacity in a range from 30 to 37 keV and another material exhibits relatively strong opacity in a range from 40 to 50 keV. Or at least one material exhibits relatively strong opacity in a range from 60 to 67 keV and another material exhibits relatively strong opacity in a range from 70 to 80 keV.

In some embodiments, the radiopaque materials include barium (Ba), bismuth (Bi), tantalum (Ta), and/or tungsten (W). For example, the radiopaque materials may include compounds containing barium, such as barium sulfate (BaSO₄); bismuth, such as bismuth oxychloride (BiOCl); tantalum, such as tantalum oxide (Ta₂O₅); and/or tungsten, such as particles of tungsten. Other suitable radiopaque materials include bismuth subcarbonate ((BiO)₂CO₃), bismuth trioxide (Bi₂O₃), tungsten carbide (WC), silver (Ag), platinum-iridium alloy, glass (such as alumino-borosilicate glass), and combinations thereof. In one embodiment, the mixture of radiopaque materials includes barium, for example BaSO₄. In one embodiment, the mixture of radiopaque materials includes barium and bismuth, for example BaSO₄ and BiOCl. In another embodiment, the mixture of radiopaque materials includes barium and tantalum, for example BaSO₄ and Ta₂O₅. In another embodiment, the mixture of radiopaque materials includes barium and tungsten, for example BaSO₄ and W.

According to an embodiment, the radiopaque filler material includes a mixture of a first radiopaque material and a second radiopaque material different from the first radiopaque material. The first and second radiopaque materials may be included at a weight ratio of 1 to 3 parts of the first radiopaque material and 1 to 3 parts of the second radiopaque material. The first and second radiopaque materials may be included at a weight ratio ranging from 2:1 to 1:2. In one embodiment, first and second radiopaque materials are included at a weight ratio of about 1:1. The mixture of radiopaque materials may include at least 25 wt-%, at least 30 wt-%, at least 40 wt-%, at least 50 wt-%, or at least 60 wt-% of the first radiopaque material. The mixture of radiopaque materials may include at least 25 wt-%, at least 30 wt-%, at least 40 wt-%, at least 50 wt-%, or at least 60 wt-% of the second radiopaque material. The mixture of radiopaque materials may include up to 75 wt-%, up to 70 wt-%, up to 60 wt-%, up to 50 wt-%, or up to 40 wt-% of the first radiopaque material. The mixture of radiopaque materials may include up to 75 wt-%, up to 70 wt-%, up to 60 wt-%, up to 50 wt-%, or up to 40 wt-% of the second radiopaque material. For example, the mixture of radiopaque materials may include from 25-75 wt-% of the first radiopaque material and from 25-75 wt-% of the second radiopaque material. In one embodiment, the mixture of radiopaque materials includes about 50 wt-% of the first radiopaque material and about 50 wt-% of the second radiopaque material.

The filler may also include other materials, such as such as compatibilizers, heat stabilizers, UV stabilizers, and combinations thereof.

The radiopaque filler materials of the present disclosure may be used in medical devices or parts of medical devices made from polymeric materials. Medical devices include those that may be used or implanted with the aid of an imaging technique for any purpose, such as diagnosis, prevention, monitoring, treatment, or alleviation of an injury, disease, or other medical condition. Examples of such medical devices include various catheters, leads, balloons, implants, tubing, etc., and parts thereof. The radiopaque filler material may be dispersed within the polymeric material that the medical device is made of. For example, the medical device may have a body made of the polymeric material, and the radiopaque filler material may be dispersed within the polymeric material throughout the body of the medical device. On the other hand, the radiopaque filler material may be dispersed within the polymeric material that makes up a part of the medical device. For example, the radiopaque filler material may be dispersed within the polymeric material in only a portion of the body, such as a tip, a shaft, a marker, or a combination thereof. In some embodiments, the radiopaque filler material and the polymeric material are present as a single layer without an additional layer of radiopaque material. In some embodiments, radiopaque filler material forms one or more bands in or on the polymeric material. For example, the polymeric material may form a tubular body of a medical device and the radiopaque filler material forms one or more bands in the tubular body. The bands may be formed transverse to the length of the tubular body or along the length of the tubular body.

In one example, the radiopaque filler material is mixed with the polymeric material, and the polymeric mixture is used to prepare the medical device or a part thereof. The polymeric mixture may be processed in any suitable way, including but not limited to mixing, compounding, blending, milling, casting, extruding, injection molding, blow molding, etc.

The polymeric mixture may be prepared into a radiopaque part or device. The radiopaque part or device may be further combined with other parts to complete the medical device. Thus, the radiopaque part or device may form all or a part of the medical device. In one embodiment, the radiopaque part is a radiopaque marker.

The polymeric material may include any polymeric material suitable for use in medical devices. Examples of suitable polymeric materials include polyethylenes, polypropylenes, polyethylene terephthalates, polyurethanes, polytetrafluoroethylenes, polyesters, polyacrylates, polyamides, polyimides, polyether sulfones, polycarbonates, polydimethyl siloxanes and silicones, isoprene/neoprene/chloroprene rubbers, and copolymers and mixtures thereof.

The amount of radiopaque filler material in the polymeric mixture may vary according to intended use and the desired attenuation level. The polymeric mixture may include the radiopaque filler material in an amount of at least 1 volume-%, at least 5 volume-%, at least 10 volume-%, at least 20 volume-%, at least 22 volume-%, or at least 25 volume-%. The polymeric mixture may include the radiopaque filler material in an amount of up to 20 volume-%, up to 28 volume-%, up to 30 volume-%, up to 32 volume-%, up to 35 volume-%, up to 40 volume-%, or up to 50 volume-%, based on the volume of the polymeric mixture. In some embodiments, the polymeric mixture includes from 20 volume-% to 40 volume-%, or from 25 volume-% to 35 volume-% of the radiopaque filler material.

The radiopaque fillers of the present disclosure may allow less filler to be used for a given level of radiopacity achieved. The mechanical properties of components made using a filled polymeric material are in part governed by the volume occupied by the filler in the polymer matrix. An increased amount (volume) of filler may result in less desirable mechanical properties, such as reduced tensile strength. However, the radiopacity of a filled polymer is affected by the weight percent of the filler, in addition to variables such as the thickness of the component and the X-ray attenuation characteristics of the filler. When using a mixture of radiopaque materials, it is feasible to achieve greater and more consistent levels of radiopacity across a particular energy range with the same combined volume content of the filler materials as using a single radiopaque filler material. A few non-limiting examples of this are given in TABLE 1 below.

TABLE 1

| | Filler | Relative radiopacity compared to |
|---|---|---|
| Filler Weight-% | Volume-% | material with 20% BaSO₄ filler |
| 20% BaSO₄ | 5.9 | 1 |
| 10% BaSO₄ + 18% BiOCl | 5.9 | 1.15 |
| 10% BaSO₄ + 19% Ta₂O₅ | 5.9 | 1.18 |

According to an exemplary embodiment, a medical device or a portion of a medical device is made from a polymeric material and a radiopaque filler material. The medical device may be a catheter, a lead, a balloon, an implant, or the like. The radiopaque filler material includes at least a first radiopaque material and a second radiopaque material that is different from the first radiopaque material. The first and second radiopaque materials may be selected from compounds containing barium (Ba), bismuth (Bi), tantalum (Ta), and/or tungsten (W). For example, the first radiopaque material may include barium (Ba) and the second radiopaque material may include bismuth (Bi), tantalum (Ta), or tungsten (W). In one embodiment, the first radiopaque material includes barium sulfate (BaSO₄) and the second radiopaque material includes tantalum oxide (Ta₂O₅). In one embodiment, the first radiopaque material includes barium sulfate (BaSO₄) and the second radiopaque material includes bismuth oxychloride (BiOCl). The radiopaque filler material may also include a third radiopaque material. The mixture of first and second radiopaque materials may include 30 wt-% to 70 wt-% of the first radiopaque material and 30 wt-% to 70 wt-% of the second radiopaque material. The mixture of first and second radiopaque materials may make up 1 vol-% to 40 vol-% of the combined polymeric and radiopaque filler material.

According to an exemplary embodiment, a catheter is made from a polymeric material and a radiopaque filler material including a mixture of barium sulfate (BaSO₄) and another radiopaque material, such as tantalum oxide (Ta₂O₅) or bismuth oxychloride (BiOCl). The barium sulfate may make up 30 wt-% to 70 wt-% (e.g., 40 wt-% to 60 wt %) of the mixture. The radiopaque filler material may make up 1 vol-% to 40 vol-% of the combined polymeric and radiopaque filler material.

According to an exemplary embodiment, a lead is made from a polymeric material and a radiopaque filler material including a mixture of barium sulfate (BaSO₄) and another radiopaque material, such as tantalum oxide (Ta₂O₅) or bismuth oxychloride (BiOCl). The barium sulfate may make up 30 wt-% to 70 wt-% (e.g., 40 wt-% to 60 wt %) of the mixture. The radiopaque filler material may make up 1 vol-% to 40 vol-% of the combined polymeric and radiopaque filler material.

According to an exemplary embodiment, a radiopaque marker is made from a polymeric material and a radiopaque filler material including a mixture of barium sulfate (BaSO₄) and another radiopaque material, such as tantalum oxide (Ta₂O₅) or bismuth oxychloride (BiOCl). The barium sulfate may make up 30 wt-% to 70 wt-% (e.g., 40 wt-% to 60 wt %) of the mixture. The radiopaque filler material may make up 1 vol-% to 40 vol-% of the combined polymeric and radiopaque filler material.

### Examples

The radiopacity of three different fillers was evaluated to compare the performance of the fillers at the two different imaging voltages. Polyurethane wedge samples with different radiopaque filler materials and a background sample without a radiopaque filler material were prepared. The first sample included bismuth oxychloride (BiOCl) as the radiopaque filler, the second sample included barium sulfate (BaSO₄) as the radiopaque filler, and the third sample included a mixture of BaSO₄ and BiOCl as the radiopaque filler in equal amounts by weight.

Fluoroscopic images of the samples were obtained at 51 kVp and at 70 kVp to compare the radiopacity of each sample at the two different voltages. The difference in the grayscale pixel density between the sample and the background was calculated to provide a measure of radiopacity. The results are given in TABLE 2 below. It should be noted that the filler loading (weight percent of sample) varied from one sample to the next, causing differences in the magnitude of the pixel density between samples. However, the differences in radiopacity are comparable between two imaging voltages.

TABLE 2

| | | Mean | |
|---|---|---|---|
| | Energy | Pixel | Pixel Density Difference |
| Filler Material | (kV) | Density | (Background − Sample) |
| Background (no filler) | 70 | 254.959 | |
| BiOCl | 70 | 221.731 | 33.2 |
| Background (no filler) | 51 | 193.331 | |
| BiOCl | 51 | 130.514 | 62.8 |
| Background (no filler) | 70 | 247.547 | |
| BaSO₄ | 70 | 206.723 | 40.8 |
| Background (no filler) | 51 | 183.363 | |
| BaSO₄ | 51 | 148.193 | 35.2 |
| Background (no filler) | 70 | 254.103 | |
| BaSO₄ + BiOCl | 70 | 218.576 | 35.5 |
| Background (no filler) | 51 | 179.279 | |
| BaSO₄ + BiOCl | 51 | 143.667 | 35.6 |

Values for the third sample (BaSO₄+BiOCl) at 70 kVp and 51 kVp show that there is no significant difference in the radiopacities at two different imaging voltages for the material containing a mixture of two fillers. However, samples containing a single filler show significant variation in the radiopacities at two imaging voltages.

Radiopaque filler materials, polymeric compositions that include the radiopaque filler materials, and devices made from the polymeric compositions are disclosed. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A medical device comprising a polymeric material and radiopaque filler material dispersed within the polymeric material, the radiopaque filler material comprising a mixture of:
a first radiopaque material; and
a second radiopaque material different from the first radiopaque material.

2. The medical device of claim 1, wherein the first and second radiopaque materials are selected from compounds containing barium (Ba), bismuth (Bi), tantalum (Ta), tungsten (W), or a combination thereof.

3. The medical device of claim 1, wherein the first radiopaque material comprises barium (Ba).

4. The medical device of claim 1, wherein the first radiopaque material comprises barium (Ba) and the second radiopaque material comprises bismuth (Bi), tantalum (Ta), or tungsten (W).

5. The medical device of claim 1, wherein the first radiopaque material comprises barium sulfate (BaSO₄) and the second radiopaque material comprises tantalum oxide (Ta₂O₅).

6. The medical device of claim 1, wherein the first radiopaque material comprises barium sulfate (BaSO₄) and the second radiopaque material comprises bismuth oxychloride (BiOCl).

7. The medical device of claim 1, wherein the first radiopaque material comprises barium sulfate (BaSO₄) and the second radiopaque material comprises tungsten (W).

8. The medical device of claim 1, wherein the first and second radiopaque material are mixed at a weight ratio of 30 to 70 parts of the first radiopaque material and 70 to 30 parts of the second radiopaque material.

9. The medical device of claim 1, wherein the first and second radiopaque material are mixed at a weight ratio of 40 to 60 parts of the first radiopaque material and 60 to 40 parts of the second radiopaque material.

10. The medical device of claim 1, wherein the mixture of first and second radiopaque materials comprises 30 wt-% to 70 wt-% of the first radiopaque material.

11. The medical device of claim 1, wherein the mixture of first and second radiopaque materials comprises 30 wt-% to 70 wt-% of the second radiopaque material.

12. The medical device of claim 1, wherein the barium sulfate (BaSO₄) comprises 50 wt-% to 60 wt-% of the mixture.

13. The medical device of claim 1, wherein the mixture of first and second radiopaque materials comprises 1 vol-% to 40 vol-% of the combined polymeric material and radiopaque filler material.

14. The medical device of claim 1, wherein at a first imaging energy output level the first radiopaque material has a first level of radiopacity and the second radiopaque material has a second level of radiopacity that is lower than the first level of radiopacity; and
wherein at a second imaging energy output level the first radiopaque material has a third level of radiopacity and the second radiopaque material has a fourth level of radiopacity that is higher than the third level of radiopacity.

15. The medical device of claim 14, wherein the radiopaque filler material has a level of radiopacity that varies no more than 20% between the first imaging energy output level and the second imaging energy output level.

16. The medical device of claim 1, wherein the mixture further comprises a third radiopaque material.

17. The medical device of claim 1, wherein the medical device comprises a catheter, a lead, or balloon.

18. The medical device of claim 1, wherein the medical device comprises a body made of the polymeric material, and wherein the radiopaque filler material is dispersed within the polymeric material throughout the body.

19. The medical device of claim 1, wherein the radiopaque filler material is substantially evenly distributed throughout the polymeric material.

20. The medical device of claim 1, wherein the radiopaque filler material forms one or more bands in the polymeric material.

21. A polymeric composition comprising a polymer and radiopaque filler material dispersed within the polymer, the radiopaque filler material comprising a mixture of:
a first radiopaque material; and
a second radiopaque material different from the first radiopaque material.

22. The polymeric composition of claim 21, wherein the first and second radiopaque materials are selected from compounds containing barium (Ba), bismuth (Bi), tantalum (Ta), and/or tungsten (W).

23. The polymeric composition of claim 21, wherein the first radiopaque material comprises barium sulfate (BaSO₄) and the second radiopaque material comprises tantalum oxide (Ta₂O₅) or bismuth oxychloride (BiOCl) or a combination thereof.

24. The polymeric composition of claim 21, wherein the first and second radiopaque material are mixed at a weight ratio of 30 to 70 parts of the first radiopaque material and 70 to 30 parts of the second radiopaque material.

25. The polymeric composition of claim 21, wherein the mixture of first and second radiopaque materials comprises 1 vol-% to 40 vol-% of the combined polymeric material and radiopaque filler material.

26. A polymeric composition comprising a polymer and a means for imparting radiopacity to the polymeric composition, the radiopacity comprising a first level of radiopacity at a first imaging energy output level and a second level of radiopacity at a second imaging energy output level, the second imaging energy output level being within 20 keV of the first imaging energy output level, the second level of radiopacity being from 90% to 110% of the first level of radiopacity, the polymeric composition exhibiting the first and second levels of radiopacity throughout the polymeric composition.

27. The polymeric composition of claim 26, wherein the means for imparting radiopacity comprises a radiopaque filler material dispersed in the polymer, the radiopaque filler material comprising a mixture of a first radiopaque material and a second radiopaque material different from the first radiopaque material.

28. The polymeric composition of claim 26, wherein the first and second radiopaque materials are selected from compounds containing barium (Ba), bismuth (Bi), tantalum (Ta), and/or tungsten (W).

29. The polymeric composition of claim 26, wherein the first radiopaque material comprises barium sulfate (BaSO₄) and the second radiopaque material comprises tantalum oxide (Ta₂O₅) or bismuth oxychloride (BiOCl) or a combination thereof.

30. The polymeric composition of claim 26, wherein the first and second radiopaque material are mixed at a weight ratio of 30 to 70 parts of the first radiopaque material and 70 to 30 parts of the second radiopaque material.

31. The polymeric composition of claim 26, wherein the mixture of first and second radiopaque materials comprises 1 vol-% to 40 vol-% of the combined polymeric material and radiopaque filler material.
